(19) United States
(12) Patent Application Publication
(10) Pub. No.: US 2004/0258775 A1
(43) Pub. Date: Dec. 23, 2004

(54) Headache, blood constipation, tapeworm, hemorrhoid, constipation and stomach acidity relief composition and method of use thereof

(76) Inventor: Sureshchandra K. Patel, Hanover Park, IL (US)

Correspondence Address: LAW OFFICES OF MARK E WIEMELT, PC, 10 SOUTH LASALLE STREET, STE. 3300, CHICAGO, IL, 60603, US

(21) Appl. No.: 10/602,029

(22) Filed: Jun. 23, 2003

(51) Int. Cl. A61K 35/78

(52) U.S. Cl. 424/745; 424/757; 424/756

(57) ABSTRACT

A chemical composition including asafetida, ajwain seed, videnga, cumin seeds, fenugreek, triphala, fennel seed, and turmeric and the method of use thereof as a treatment for relieving headache, blood constipation, tapeworm, hemorrhoid, constipation and stomach acidity symptoms.

### BACKGROUND OF THE INVENTION

1. Field of the Invention

This present invention relates to a composition which relieves headache, blood constipation, tapeworm, hemorrhoid, constipation and stomach acidity symptoms. The new composition has increased therapeutic efficacy. The invention particularly relates to a pharmaceutical composition containing asafetida, tumeric, and other compounds as will be shown. The invention also relates to methods of use of the chemical composition.

2. Description of the Related Art

The quest for newer drugs, dosage forms or better formulations is motivated by an overriding requirement, e.g. minimum possible dose, in a dosage form that will provide controlled, reproducible therapeutic amounts of the drug to the body. The bioavailability is, therefore, an essential part of the drug formulation and has come to be increasingly accepted. In other words it means that performance of the drug in vivo is the most important factor in order to evaluate its efficacy.

A look into the Indian system of medicine shows that generally complex formulations are prescribed for almost all ailments. For this matter certain herbs, either in groups or individually, occupy a very prestigious position in the majority of these prescriptions. Some of these groups are:

TRIKATU: Piper nigrum, Piper longum & Zingiber officinalis in equal proportion W/W.

PANCKOL: Piper longum (roots), Piper longum (fruits), Piper Chaba (roots), Plumbago zeylanica.

UMBELLIFEREAE—Herbs: Carum carvi, Anethum graveolans, Anethum Sowa.

As examples of the individual herbs Acorus calamus (Arcidate) and Semicarpus anacardium (Anacardiaceae) have been reported frequently in large number of Ayurvedic formulations. Powdered herbs are combined with other ingredients of the formulations to increase their potency and therapeutic efficacy.

Out of the all the herbs mentioned above TRIKATU or its individual constituents especially Piper nigrum and Piper longum are the most widely used herbs in Ayurveda. The literature pertaining to the use of these herbs dates back to the period between 6th century Ad and 3rd century BC (Charaka, et al, Charak Samhita, 3rd edn, Nirnaya Sagar Press Bombay, 1941 (in Sanskrit), Kaviraj, K. B., Sushruta Samhita, 2nd ed., Chowkhamba Sanskrit Series, vol. 3, Varanasi, India 1953; Vagbmat, Ashtang Hridaya, Chowkhamba Sanskrit Series, Varanasi, India, 1962 (in Sanskrit). Hand Book of Domestic Medicine and Common Ayurvedic Remedies (1979) published by Central Council of Research in Indian Medicine and Homeopathy, New Delhi in India reveals that out of 370 compound formulations listed therein, 210 contain either Trikatu or its individual ingredients. If the claim made about the individual members of the Trikatu group are examined in literature, it is amazing to find that the single entity have been shown to be panacea for numerous ailments. One, therefore, wonders whether these herbs have some inherent properties to cure a variety of the aliments or a role other than their therapeutic action.

Asafetida, also called Food of the Gods and Devil's Dung, is a gum resin obtained from the plant Feritla asafetida. Prior uses of asafetida have been as a sedative and as a carminative to relieve colic and promote gas expulsion from the gastrointestinal tract. Asafetida has also been previously used as a drug to relieve convulsions and spasms, as a treatment for nerve disorders, as a stimulant to the brain and nervous system, and as a treatment for skin allergies.

Another spice used as a remedy is Cumin seed. Cumin seed, Cuminum cyminum, has effectively been used as an anti-gas remedy and as a remedy for dyspeptic headache. Yet another spice is fenugreek. Fenugreek, Trigonella foenum-graecum, has been used in preventing fevers and alleviating stomach indigestion.

Yet another spice is triphala. Triphala is comprised of three fruits, namely 1) Harada, Chebulic myrobalan, also known as Terminalia chebula, 2) amla, Emblic myrobalan, also known as Emblica officinalis, and 3) behada, Beleric myrobalan, also known as Terminalia belerica. Triphala is a known laxative, antioxidant, a powerful eye rejuvenator for treatment of conjunctivitis, and an anti-inflammatory agent.

Terminalia chebula, has been used to promote gastrointestinal mobility. Terminalia belerica has been used to treat excess mucus, asthma, bronchiole conditions and to treat allergies. Emblica officinalis has been used to alleviate symptoms associated with fever, liver disorder, indigestion, anemia, heart complaints and urinary problems. Fennel seed, Foeniculum vulgare, has been used to alleviate symptoms associated with angina, high blood pressure, asthma, body odor, promote lactation, heartburn, abdominal pain, colon disorders, gas, gastrointestinal spasms and stomach acidity.

Another spice used as a remedy is turmeric. The rhizome from Curcuma longa is harvested and ground to make the turmeric spice. Prior uses of turmeric have been used to treat symptoms associated with indigestion, rheumatic pains, poor vision, coughs, and to increase milk production. The active constituent of turmeric is curcumin. Curcumin has been shown to have a wide range of therapeutic actions. First, it protects against free radical damage because it is a strong antioxidant. Sreejayan N, Rao MNA. Free radical scavenging activity of curcuminoids. Arzneimittelforschung 1996;46: 169-71; Ramirez-Boscá A, Soler A, Gutierrez MAC, et al. Antioxidant curcuma extracts decrease the blood lipid peroxide levels of human subjects. Age 1995;18:167-9. Second, it reduces inflammation by lowering histamine levels and possibly by increasing production of natural cortisone by the adrenal glands. Arora R B, Basu N, Kapoor V, Jain A P. Anti-inflammatory studies on Curcuma longa (turmeric). Ind J Med Res 1971;59:1289-95. Third, it protects the liver from a number of toxic compounds. Kiso Y, Suzuki Y, Watanbe N, et al. Antihepatotoxic principles of Curcuma longa rhizomes. Planta Med 1983;49:185-7.

Another spice is Carum copticum, known as Ajwain seed. Ajwain seed is a known to promote digestion, alleviates gas and promotes kidney function. Embelia ribes, known as vidanga, works to expel intestinal worms, prevents flatulence and is useful is dyspepsia.

There is a need in the art for a method for relief of headache, blood constipation, tapeworm, hemorrhoid, constipation and stomach acidity symptoms. There is also a need in the art for a method for administering the chemical composition is capsule form. There is also a need in the art for combining these chemical compositions in a capsule form.

It is a primary object of the instant invention to provide a method for relieving headache symptoms.

It is a primary object of the instant invention to provide a method for relieving blood constipation symptoms.

It is a primary object of the instant invention to provide a method for relieving symptoms associated with tapeworm.

It is a primary object of the instant invention to provide a method for relieving symptoms associated with hemorrhoids.

It is a primary object of the instant invention to provide a method for relieving symptoms associated with constipation.

It is a primary object of the instant invention to provide a method for relieving stomach acidity symptoms.

A further object of the present invention is to provide a method for relieving headache symptoms, blood constipation symptoms, symptoms associated with tapeworm, symptoms associated with hemorrhoids, symptoms associated with constipation, stomach acidity symptoms in capsule form.

Another object of the instant invention is to provide a low cost remedy for the relief of headache symptoms, blood constipation symptoms, symptoms associated with tapeworm, symptoms associated with hemorrhoids, symptoms associated with constipation, stomach acidity symptoms.

These together with other objects of the invention, along with the various features of novelty which characterize the invention, are pointed out with particularity in the detailed description annexed to and forming a part of this disclosure. For a better understanding of the invention, its operating advantages and the specific objects attained by its uses, reference should be had to the descriptive matter in which is illustrated a preferred embodiment of the invention.

### BRIEF SUMMARY OF THE INVENTION

According to the invention there is provided a chemical composition comprising asafetida, ajwain seed, vidanga, cumin seeds, fenugreek, triphala, fennel seed, and turmeric which is to be combined in capsule form to relieve headache, blood constipation, tapeworm, hemorrhoid, constipation and stomach acidity symptoms.

There has thus been outlined, rather broadly, the more important features of the invention in order that the detailed description thereof that follows may be better understood, and in order that the present contribution to the art may be better appreciated. There are, of course, additional features of the invention that will be described hereinafter and that will form the subject matter of the invention. Those skilled in the art will appreciate that the conception, upon which this disclosure is based, may readily be utilized as a basis for other methods for carrying out the several purposes of the present invention. It is important, therefore, that the invention be regarded as including such equivalent constructions insofar as they do not depart from the spirit and scope of the present disclosure.

### DETAILED DESCRIPTION OF THE PREFERRED EMBODIMENT

While the invention may be susceptible to embodiments in different forms, there will be described in detail specific embodiments, with the understanding that the present disclosure is to be considered an exemplification of the principles of the invention, and is not intended to limit the invention to that as illustrated and described herein.

The chemical composition of the preferred embodiment of this invention includes asafetida, ajwain seed, vidanga, cumin seeds, fenugreek, triphala, fennel seed, and turmeric. No one has yet discovered the extreme effectiveness of this chemical composition as a relief for symptoms associated with headache, blood constipation, tapeworm, hemorrhoid, constipation and stomach acidity until this invention. This is a new and novel use for this material.

The preferred embodiment is placed within a capsule using known techniques, however the composition may be mixed together and formed into a tablet, or administered in syrup or dry powder form. In the preferred embodiment, each chemical component is taken in dry powder form in a proportion as will be shown. The components are then preferably mixed together along with water and boiled for approximately thirty minutes. The resulting composition is then allowed to cool and placed within a gel capsule using known techniques.

The following example is intended to illustrate the invention without limiting the invention. The preferred chemical composition of the capsule is 5 milligrams (mg) of asafetida, 100 mg of ajwain seed, 50 mg of vidanga, 20 mg of cumin seeds, 20 mg of fenugreek, 100 mg of triphala, 180 mg of fennel seed, and 25 mg of turmeric. The 100 mg of triphala preferably contains 15 mg Terminalia chebula, 35 mg of Terminalia belerica, and 50 mg of Embelica officinalis. However, equal portions of each may be used.

The chemical composition is to be applied orally by capsule. Two capsules administered every morning for 30 days helps relieve the aforementioned symptoms. While the invention has been described in connection with a preferred embodiment it will be understood that it is not intended that the invention be limited to that embodiment. On the contrary, it is intended to cover all alternatives, modifications and equivalents as may be included within the spirit and scope of the invention as disclosed.

As to the manner and usage and operation of the instant invention, same should be apparent from the above disclosure, and accordingly no further discussion relevant to the manner of usage and operation of the instant invention shall be provided.

With respect to the above description then, it is to be realized that the modifications of this chemical composition of this invention are deemed readily apparent and obvious to one skilled in the art, and all equivalent relationships are intended to be encompassed by the present invention.

Therefore, the foregoing is considered illustrative of only the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

The foregoing discussion is illustrative of the invention. However, since many embodiments of the invention can be made without departing from the spirit and scope of the invention, the invention resides wholly in the claims hereinafter appended.

I claim:

1. A chemical composition comprising asafetida, ajwain seed, vidanga, cumin seeds, fenugreek, triphala, fennel seed, and turmeric.

2. The chemical composition of claim 1 wherein the composition contains approximately 5 milligrams of asafetida.

3. The chemical composition of claim 1 wherein the composition contains approximately 100 milligrams of ajwain seed.

4. The chemical composition of claim 1 wherein the composition contains approximately 50 milligrams of vidanga.

5. The chemical composition of claim 1 wherein the composition contains approximately 20 milligrams of cumin seeds.

6. The chemical composition of claim 1 wherein the composition contains approximately 20 milligrams of fenugreek.

7. The chemical composition of claim 1 wherein the composition contains approximately 100 milligrams of triphala.

8. The chemical composition of claim 1 wherein the composition contains approximately 180 milligrams of fennel seed.

9. The chemical composition of claim 1 wherein the composition contains approximately 25 milligrams of turmeric.

10. A chemical composition comprising comprising approximately 5 milligrams of asafetida, approximately 100 milligrams of ajwain seed, approximately 50 milligrams of vidanga, approximately 20 milligrams of cumin seeds, approximately 20 milligrams of fenugreek, approximately 100 milligrams of triphala, approximately 180 milligrams of fennel seed, and approximately 25 milligrams of turmeric.

11. A method of relieving headache, blood constipation, tapeworm, hemorrhoid, constipation and stomach acidity symptoms comprising orally administering a composition containing asafetida, ajwain seed, vidanga, cumin seeds, fenugreek, triphala, fennel seed, and turmeric.

12. The method of claim 1 wherein the composition contains approximately 5 milligrams of asafetida.

13. The method of claim 1 wherein the composition contains approximately 100 milligrams of ajwain seed.

14. The method of claim 1 wherein the composition contains approximately 50 milligrams of vidanga.

15. The method of claim 1 wherein the composition contains approximately 20 milligrams of cumin seeds.

16. The method of claim 1 wherein the composition contains approximately 20 milligrams of fenugreek.

17. The method of claim 1 wherein the composition contains approximately 100 milligrams of triphala.

18. The method of claim 1 wherein the composition contains approximately 180 milligrams of fennel seed.

19. The method of claim 1 wherein the composition contains approximately 25 milligrams of turmeric.

20. A method of relieving symptoms associated with headaches, blood constipation, tapeworm, hemorrhoids, constipation, and stomach acidity comprising orally administering a composition containing approximately 5 milligrams of asafetida, approximately 100 milligrams of ajwain seed, approximately 50 milligrams of vidanga, approximately 20 milligrams of cumin seeds, approximately 20 milligrams of fenugreek, approximately 100 milligrams of triphala, approximately 180 milligrams of fennel seed, and approximately 25 milligrams of turmeric.
